(19) United States
(12) Patent Application Publication
(10) Pub. No.: US 2016/0051541 A1
(43) Pub. Date: Feb. 25, 2016

(54) PHARMACEUTICAL COMPOSITIONS COMPRISING VANOXERINE AND ANTIANGINAL COMPOUNDS, AND METHODS OF ADMINISTRATION OF THE SAME FOR TREATING EPISODES OF CARDIAC ARRHYTHMIA, MAINTAINING NORMAL SINUS RHYTHM, PREVENTING RECURRENCE OF CARDIAC ARRHYTHMIA, AND TREATMENT OF CHRONIC CARDIAC ARRHYTHMIA IN MAMMALS

(71) Applicant: CHANRX CORPORATION, Cleveland, OH (US)

(72) Inventors: Arthur M. Brown, Brecksville, OH (US); Howard C. Dittrich, La Jolla, CA (US)

(21) Appl. No.: 14/781,296

(22) PCT Filed: Apr. 25, 2014
(86) PCT No.: PCT/US14/35569

§ 371 Date: Sep. 29, 2015

Related U.S. Application Data
(60) Provisional application No. 61/816,214, filed on Apr. 26, 2013.
(60) Provisional application No. 61/905,054, filed on Nov. 15, 2013.
(60) Provisional application No. 61/904,753, filed on Nov. 15, 2013.
(60) Provisional application No. 61/915,792, filed on Dec. 13, 2013.

(51) Int. Cl. A61K 31/495

(52) U.S. Cl. CPC A61K 31/495

(57) ABSTRACT

Disclosed embodiments are related to pharmaceutical compositions comprising vanoxerine and an antianginal compound and methods of administration of vanoxerine and an antianginal composition to maintain steady state pharmacological concentration levels in a mammal.

### FIELD OF THE INVENTION

Presently disclosed embodiments are related to compositions comprising vanoxerine and an antianginal compound, and methods of treatment comprising administration of vanoxerine in conjunction with the antianginal compound for terminating acute episodes of cardiac arrhythmia. Presently disclosed embodiments particularly relate to methods for dosing and treatment methodologies for administration of vanoxerine in the case of chronic cardiac arrhythmia.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732.) Vanoxerine has also been used for treating and preventing cardiac arrhythmia in mammals. (See U.S. Pat. No. 6,743,797 and U.S. Pat. No. 7,700,600.)

It is desirable to optimize compositions for treatment of cardiac arrhythmia in conjunction with compounds that support the consistent use of vanoxerine for prevention of cardiac arrhythmia, maintenance of sinus rhythm, and prevention of recurrence of cardiac arrhythmia, and methods of treatment using vanoxerine in conjunction with an antianginal agent that provides for regular administration of vanoxerine or concomitant administration of vanoxerine and an antianginal compound for treatment of cardiac arrhythmias.

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of recurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (T_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two Components of Cardiac Delayed Rectifier K+Current. Differential Sensitivity to Block by Class III Anti-Arrhythmic Agents,”J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2,4′-piperidin]-6y1], (+)-, monochloride (MK-499) predominantly, if not exclusively, block I. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S 17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supraventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

Therefore, it is necessary to develop compositions that provide for safe and efficacious long term use of vanoxerine and methods of using the same for treatment of cardiac arrhythmia, wherein the compositions and methods comprise vanoxerine and one additional anti-anginal compound.

### SUMMARY

Embodiments of the present disclosure relate to compositions for treating cardiac arrhythmias comprising vanoxerine and one or more anti-anginal compound and a pharmaceutical carrier.

Further embodiments of the present disclosure relate to compositions for treating cardiac arrhythmias comprising vanoxerine and one or more compounds altering sodium-dependent calcium channels and a pharmaceutical carrier.

Further embodiments of the present disclosure relate to methods for treating cardiac arrhythmias comprising: administering a composition comprising vanoxerine and one or more anti-anginal compounds to a mammal for the treatment of cardiac arrhythmia.

Further embodiments of the disclosure include a pharmaceutical composition comprising an effective amount of vanoxerine, an anti-anginal compound, and a pharmaceutical carrier, which is suitable for administration to a mammal for treatment of cardiac arrhythmia.

Further embodiments of the present disclosure relate to methods for treating cardiac arrhythmias comprising: administering a composition comprising vanoxerine and ranolazine to a mammal for the treatment of cardiac arrhythmia.

Further embodiments of the present disclosure relate to methods for treating cardiac arrhythmias comprising: administering to a mammal an effective amount of a first dose of a composition comprising vanoxerine and ranolazine, a second dose of a composition comprising ranolazine for treatment of cardiac arrhythmias; wherein subsequent administration alternates between a composition with and without vanoxerine.

Further embodiments of the present disclosure relate to methods for treating cardiac arrhythmias comprising: administering to a mammal alternating doses of an effective amount of a first dose of ranolazine and an effective amount of a second dose comprising vanoxerine and ranolazine for treatment of cardiac arrhythmias.

Other aspects of the present disclosure are directed to methods for preventing a recurrence of an episode of cardiac arrhythmia in a mammal, such as a human, by administering to that mammal at least an effective amount of a first composition comprising ranolazine, administering to said same patient an effective amount of a second composition comprising vanoxerine.

Other aspects of the present disclosure are directed to methods for preventing a recurrence of an episode of cardiac arrhythmia in a mammal, such as a human, by administering to that mammal at least an effective amount of a first composition comprising an anti-anginal drug and, about every 48 hours, administering to said same mammal, an effective amount of a composition comprising vanoxerine.

Other aspects of the present disclosure are directed to methods for preventing a recurrence of an episode of cardiac arrhythmia in a mammal, such as a human, by administering to that mammal at least an effective amount of a first composition comprising an anti-anginal drug, and, about every 72 hours, administering to said same mammal, an effective amount of a composition comprising vanoxerine.

Other aspects of the present disclosure are directed to methods for preventing a recurrence of an episode of cardiac arrhythmia in a mammal, such as a human, by administering to that mammal at least an effective amount of a first composition comprising an anti-anginal drug, and, about every 96 hours, administering to said same mammal, an effective amount of a composition comprising vanoxerine.

A further embodiment of the present disclosure includes an additional step in any of the foregoing methods wherein said first step comprises administering vanoxerine to establish steady state in a mammal and administering to said mammal a second anti-arrhythmic composition subsequent to establishment of said steady state, and further comprising subsequent administration of vanoxerine to maintain said steady state.

Accordingly, a further embodiment is directed to a method for preventing a re-occurrence of an episode of cardiac arrhythmia in a mammal, such as a human, by administering to that mammal at least an effective amount of vanoxerine to induce steady state, and then administering a further composition comprising an anti-anginal drug having a dosing schedule of about every 72, 48, 36, 24, 16, 12, 8, or 4 hours or for example, every fourth day, every third day, every other day, every day, or two, three, or four times daily and, further administering to said same mammal, an effective amount of a composition comprising vanoxerine to maintain said steady state.

A method of chronic administration of vanoxerine comprising a loading phase and a maintenance phase, wherein said loading phase comprising administration of about 25 to 200 mg of vanoxerine daily until steady-state concentrations are met; and wherein said maintenance phase comprises administration of vanoxerine to maintain said steady-state concentration, and wherein said maintenance phase further comprises administration of at least one additional anti-arrhythmic drug administered to said same patient.

A method of treating a patient suffering from cardiac arrhythmia comprising a loading phase and a maintenance phase: identifying a patient experiencing an episode of cardiac arrhythmia; during the loading phase, administering vanoxerine to said patient for induction of steady state pharmacological concentration; and during the maintenance phase, administering an antianginal compound subsequent to induction of steady state pharmacological concentration, and administering a further dose of vanoxerine to said patient to maintain steady state pharmacological concentration.

Administering steps in any of the foregoing methods may comprise administration by a caregiver, a medical professional, or self-administered by a patient.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “CYP3A4” means the cytochrome P450 3A4 protein, which is a monooxygenase that is known for its involvement in drug metabolism.

As used herein, “administering” or “administer” refers to the actions of a medical professional or caregiver, or alternatively self-administration by the patient.

As used herein, “antianginal” means any drug used in the treatment of angina pectoris, or chest pain due to ischemia of the heart muscle.

The term “alternating dosing routine” means a dosing routine wherein two or more drugs are taken in a standard routine, wherein each drug follows a standard but different dosing routing from at least one other drug. For example, administration of a first pill comprising drugs A and B on day one, a second pill comprising only drug B on day 2 and 3, followed by a pill comprising drugs A and B on the fourth day and repeating. This concept can include numerous designs, lengths, etc., but is intended to allow for administration of different drugs, or different doses of one or more drugs in a dosing routine.

The term “monophasic dosing routine” means dosing of a single dose of a given drug over a period.

The term “multiphasic dosing routine” means a dosing regimen wherein varying doses are provided of one or more drugs over a given period.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardria, and ventricular fibrillation, and combinations thereof are all capable of severe morbidity and death if left untreated. Methods and compositions described herein are suitable for the treatment of these and other cardiac arrhythmias.

Interestingly, studies have identified that human subjects have significant variability with regard to the metabolism of vanoxerine. Vanoxerine, is susceptible to metabolism by CYP3A4 among other known P450 cytochromes. Accordingly, the bioavailability of a given dose of vanoxerine is impacted by certain P450 cytochromes. In particular, studies have identified that human subjects have variability with regard to metabolism which is predicted to be based on CYP3A4 and other P450 cytochromes. Typically, patients fall within one of two groups, a fast metabolism or a slow metabolism, such that the patients can be grouped with other patients and will have similar metabolic profiles for a given dose of vanoxerine. Patients in the fast metabolism group respond differently to vanoxerine than patients in the slow metabolism group with regard to Cₘₐₓ, tₘₐₓ and AUC plasma concentrations as well as the half-life. Accordingly, it is possible to define whether a given patient is a fast or a slow metabolizer and predict their pharmacokinetic response to vanoxerine. Accordingly, determination of the patient's status within the fast or slow metabolic group can be utilized for improving efficacy and treatment of a patient.

Additionally, patients fall within a gradient within the slow and fast metabolism groups. Accordingly, there exists, even within the groupings, a continuum that provides that some people are faster or slower metabolizers even within the groups. Additional factors also play into the variability with regard to patient populations. Accordingly, when providing efficacious treatment for termination of cardiac arrhythmias, in some embodiments, it is important to determine or recognize where the patient falls within the spectrum of vanoxerine bioavailability, and provide a dose of vanoxerine that will be efficacious for that patient while also maximizing the safety profile of the drug.

Vanoxerine also has a moderately low oral bioavailability as a result of incomplete absorption and substantial first pass metabolism, from CYP3A4 and other p450 inhibitors. Vanoxerine is primarily eliminated from the body in urine, bile, and feces. Indeed, a substantial amount of the drug is expelled unabsorbed into the feces. Additionally, pharmacokinetic parameters from tests in dogs suggest that there is a slow Tₘₐₓ of about 3 hours, low systemic bioavailability (23%) and slow elimination from the plasma (T_{1/2} of 22 hours). However, the long half-life of the drug may actually be utilized to minimize the continuous or regular dosing of the drug.

Further studies have questioned whether sustained, and/or chronic use of vanoxerine is suitable for mammalian patients. Preliminary studies have suggested that daily use of a drug over 7, 10, and 14 days may lead to increased heart rate and systolic blood pressure when taking concentrations of 75, 100, 125, and 150 mg of vanoxerine a day. However, control and prevention of events of cardiac arrhythmia are important to these patients to prevent future recurrences and the deleterious effects and morbidity.

Indeed, control and prevention of events of cardiac arrhythmia are important to these patients to prevent future recurrences and the deleterious effects and morbidity. One issue is that cardiac arrhythmia is a progressive disease and patients that suffer from a first cardiac arrhythmia are pre-disposed to suffering from additional episodes of cardiac arrhythmia. Any cardiac arrhythmia involves risk with regard to mortality and morbidity, and so terminating the cardiac arrhythmia in a timely and safe manner is a critical need for these patients. Therefore, preventing further arrhythmic events is paramount for limiting this risk.

Therefore, upon an occurrence of cardiac arrhythmia, patients often visit an emergency room or other medical provider for administration of certain drugs that treat the cardiac arrhythmia, or other treatments, including ablation. However, it is not always feasible to quickly reach a doctor for fast, safe, and effective treatment of cardiac arrhythmia. Furthermore, in view of the dangers of some concomitant administration with other drugs, it is advantageous to provide patients who have previously suffered from a cardiac arrhythmia, and have successfully treated that cardiac arrhythmia with vanoxerine, with a combination drug and a method of administration that provides for a maintenance of vanoxerine in the body, while concomitantly taking a further anti-arrhythmic drug so as to prevent recurrence of cardiac arrhythmias.

Additional concerns for patients who have suffered from cardiac arrhythmia is compounding heart disease, angina pectoris, as well as other heart pain, chest pain, and other complications. Typically, concomitant use of an atrial fibrillation drug with a number of other drugs is contraindicated because of any number of interactions between the two drugs. However, certain drugs may establish a beneficial co-administration with vanoxerine wherein the concomitant administration of vanoxerine and at least one additional drug for treatment of cardiac arrhythmia allows for maintenance of steady state status of vanoxerine while providing for more frequent administration of said at least one additional drug. The combination allows for regular administration of vanoxerine to maintain normal sinus rhythm, but without the need for daily maintenance therapy, while providing for a dose of a second drug to be taken more frequently than the vanoxerine, and aiding in the maintenance of normal sinus rhythm, and preventing further episodes of cardiac arrhythmia.

In view of the concerns of concomitant administration, and in view of concerns about repeated dosing of vanoxerine, embodiments described herein provide for administration of drugs in particular for concomitant use of vanoxerine with an anti-anginal drug. In particular embodiments, the anti-anginal drug is a sodium-dependent calcium channel compound such as ranolazine. Ranolazine has been shown to produce atrial-selective depression of sodium channel-dependent parameters and to suppress atrial fibrillation in a variety of experimental models and conditions. Certain studies have compared ranolazine with propafenone to determine the electrophysiological and anti-AF effects of propafenone and ranolazine at clinically relevant conditions in canines. These studies have shown that propafenone and ranolazine both suppress atrial fibrillation, but ranolazine, unlike propafenone suppresses the atrial fibrillation with minimal effects on ventricular myocardium, suggesting a reduced potential for promoting ventricular arrhythmias.

Any concomitant use of medications for treatment of angina include nitroglycerin, beta blockers, and calcium channel blockers, inhibitors, ACE inhibitors, statins, angiotensin-converting enzyme inhibitors, ranolazine, l-arginine, nitrates, and fatty acid oxidation inhibitors, among others.

Vanoxerine has a relatively long plasma half-life of about 22 hours, and further tests suggest that repetitive dosing in dogs provides a half-life is considerably longer at about 66 hours. Further studies have suggested that the half-life may extend up to 125 hours in some cases. These studies have reported that in some cases steady state is achieved within 3 days of oral dosing. Indeed, tests on recovery of administration of radioactivity labeled vanoxerine in rats were incomplete. This, coupled with the observed biliary excretion, suggests enterohepatic circulation may be occurring. This provides for an opportunity to achieve steady state plasma levels for restoration or maintenance of normal sinus rhythm in mammals.

Target plasma level concentrations, taken at a time point of 1 hour post administration are about 5 to about 1000 ng/ml. In alternative embodiments, physiological concentrations, as measured in the plasma at a time of 1 hour post administration are about 20 to about 400 ng/ml, or about 20 to about 200 ng/ml, or about 25 to about 150 ng/ml or about 40 to about 125 ng/ml, or about 60 to about 100 ng/ml. In measuring plasma levels for confirmation of half-life and/or steady state plasma levels, it may be necessary to take additional plasma level measurements at further time points, such as 2, 4, 6, 8, 12, 24, 36, 48, 72, hours, and other times as appropriate. In some cases, it may be advantageous to test plasma levels every 24, 48, 72, or 96 hours, or to test plasma levels prior to or subsequent to a further administration of vanoxerine.

To reach these concentrations, in some embodiments, a dosage of 1 mg to 1000 mg vanoxerine per unit dose is appropriate. Other embodiments may utilize a dosage of about 25 mg to 500 mg, or about 25 to 400 mg, or about 50 mg to about 400 mg, or about 200 to about 400 mg. Preferred embodiments include administration of vanoxerine in about 25, 50, 75, 100, 125, 150, 200, 300, and 400 mg doses for daily dosing or a loading period and for maintenance amounts for treatment of chronic cardiac arrhythmia. However, typically, after a loading period, lower doses of vanoxerine may be administered to maintain normal sinus rhythm.

Accordingly, it is advantageous to provide a composition that combines vanoxerine and another drug, such as an antianginal drug, that provides for consistent dosing of vanoxerine to prevent recurrence of cardiac arrhythmia without over administration of vanoxerine that may increase heart rate or blood pressure.

Such compositions may be advantageously used in methods described herein. In particular, because of the long half-life of vanoxerine, it may be advantageous to administer an anti-anginal compound on a regular schedule of daily or twice daily dosing while concomitantly administering vanoxerine less frequently based on the extended half-life of the vanoxerine, so as to maintain the concentration of vanoxerine in the patient, which thereby supports chronic use of vanoxerine. Accordingly, an embodiment of the invention comprises an anti-anginal and vanoxerine in a single dose, or taken as two individual doses for concomitant use.

In further embodiments, with regular administration of an antianginal drug, vanoxerine may be administered once daily, every other day, every third day, every fourth day, or less frequently such as once a week. In other embodiments, a method may comprise administration of an antianginal drug alone, and the antianginal drug concomitantly with vanoxerine on the second dose, and repeating, so that every second dose comprises a concomitant administration of the two compounds. There are many ways to aid in such administration, including blister packs and similar packaging. Accordingly, the packaging would contain an antianginal pill followed by a combination antiantingal and vanoxerine as a subsequent pill, which would repeat.

In other embodiments, a method comprises administration of a first dose of an antianginal drug, a second dose of an antianginal dose, and a third dose comprising an antianginal and vanoxerine, wherein the administration of the three doses is then repeated. Where an antianginal drug is taken 3 times a day, this will result in administration of vanoxerine once a day. Where an antianginal drug is taken twice daily, this will result in administration of vanoxerine every other day. Where an antianginal is taken once a day, this results in administration of vanoxerine every third day, etc.

Other embodiments may further modify the dosing regimen, wherein the vanoxerine is concomitantly administered every fourth dose, every fifth dose, every sixth dose, or every seventh dose. Again, facilitating such dosing schedules may be accomplished by any means known in the art. Typically, this includes blister packages, set in weekly or monthly type packages. As individual doses and individual schedules may depend on a particular individual's pharmacokinetic response, it is envisioned that one patient may need a dose every third day, where another might need a dose of vanoxerine every fourth day, or that the amounts may differ based on the individual.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

For oral administration, a suitable composition containing vanoxerine and or compositions comprising vanoxerine and/or an antianginal compound as disclosed herein, or a pharmaceutically acceptable salt thereof, may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g., gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine, or vanoxerine and/or an anti-anginal compound as disclosed herein, or a pharmaceutically acceptable salt thereof, and the desired additives may be filled into a capsule, such as a hard or soft gelatin capsule. The contents of a capsule and/or caplet may also be formulated using known methods to give sustained release of the active compound.

Liquid oral dosage forms of vanoxerine and vanoxerine and/or an anti-anginal compound as disclosed herein, or a pharmaceutically acceptable salt thereof, may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing vanoxerine and vanoxerine and/or an anti-anginal compound as disclosed herein, or a pharmaceutically acceptable salt thereof, may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine and vanoxerine and/or an anti-anginal compound as disclosed herein, or a pharmaceutically acceptable salt thereof, may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspensions may be prepared from sterile powders or granules having one or more carriers or diluents mentioned for use in the formulations for oral administration. Parenteral administration may be through intravenous, intradermal, intramuscular or subcutaneous injections.

In some embodiments, maintenance of a predetermined plasma level is achieved through dosing where the vanoxerine drug is administered once a day, once every other day, once every third day, once every 4^{th}, 5^{th}, 6^{th}, and 7^{th} days, wherein an additional drug is administered between vanoxerine administrations. In certain embodiments, the steady state levels are a mean plasma concentration of about 10 to about 200 ng/ml, and preferably about 20 to about 150 ng/ml, about 25-125 ng/ml, or about 50 to about 150 ng/ml.

Accordingly, in some embodiments, it is advantageous to have a loading phase of vanoxerine, wherein a mammal is given a sufficient number of doses of vanoxerine to achieve steady-state status which provides an increased half-life of about 66 hours and up to 125 hours in some cases. Upon reaching steady-state, and the resulting increased half-life, the loading phase is complete. The next phase is the maintenance phase, wherein subsequent doses of vanoxerine are administered to maintain a pre-determined steady state plasma level (or as measured in some other bodily fluid) concentrations of vanoxerine for restoration or maintenance of normal sinus rhythm in a mammal. Accordingly, the subsequent doses of vanoxerine may be given in as a single daily dose (lower does than the loading phase), or less frequently, but still maintain a steady-state pharmacological concentration in the mammal. In view of the extended half-life, such administration may be as frequent as daily, or extend to 36, 48, 72, 96, 108, 120, 125, or 144 hours or longer, as appropriate.

It may be advantageous to test a patient to determine their particular metabolic profile with regard to vanoxerine and compare their profile to known profiles of patients to determine their theoretical steady state level and time to reach such level. Indeed, as patients are slow and fast metabolizers with regard to vanoxerine, the determination of whether a patient is a slow or fast metabolizer can be utilized to appropriately determine their required dosing to reach steady state concentrations, the dose needed, and the dose needed for maintenance of the steady state levels. Therefore, a further embodiment comprises the step of administering vanoxerine to a patient, measuring their pharmacokinetic response to the vanoxerine, comparing their pharmacokinetic response to a known metabolic profile; determining whether the patient is a slow or fast metabolizer of vanoxerine, adjusting the dose to aid in the patient reaching steady state levels, and adjusting the dose given to said patient to maintain steady state levels. The adjustments are only necessary where a change is needed for improving efficacy and safety of the treatment.

It may be advantageous to further utilize a method of loading vanoxerine to achieve a steady state plasma level in connection with an additional pharmaceutical composition, wherein the vanoxerine is first administered to a mammal to reach a predetermined plasma level and steady state status, upon reaching such plasma level at a pre-determined point subsequent to the vanoxerine administration, a different drug compound is given, such as an antianginal compound which is taken daily or as indicated. Then Vanoxerine, because of the long half-life created through the steady state status, may then be taken about every 48 to 72 hours, so as to maintain the pre-determined plasma level. In preferred embodiments the antianginal compound is ranolazine.

Accordingly, a preferred course of treatment includes a first administration of vanoxerine, measuring of the pharmacokinetic response in the patient (such as measuring plasma concentration levels), determining the metabolic profile of the patient and assessing whether a modification of the dose, or duration of dosing of vanoxerine is necessary to achieve steady state concentrations. Upon reaching steady state concentrations, providing a further course of treatment that provides for a pre-determined dose of vanoxerine given about every 48-72 hours to maintain said steady state concentration, and providing for a further course of a further anti-anginal drug that is taken as indicated, for example daily, thereby providing for a maintained vanoxerine concentration in the body and concentrations of a further anti-anginal drug so as to maintain normal sinus rhythm and to prevent recurrence of cardiac arrhythmia in the patient.

In other embodiments, it is advantageous to provide for a certain dose, or a maximum dose at a given time point after administration of the vanoxerine to safely and effectively treat the cardiac arrhythmia. Accordingly, modification of Cₘₐₓ and tₘₐₓ is appropriate to maintain consistent Cₘₐₓ plasma level concentrations for a particular patient. Cₘₐₓ concentrations are about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration. These maximum values vary widely by patient and modification of the dose, the dosing schedule, diet, and other concomitant medications may be utilized to reach a predetermined therapeutic level.

In further embodiments, upon reaching a given Cₘₐₓ, it is then advantageous to provide a subsequent dose of vanoxerine that may be administered once daily, every other day, or every third day, so as to maintain pharmacological concentration of vanoxerine in the body.

There are many ways to aid in administration of varying doses to aid a patient in taking the correct dose at the correct time including blister packs and similar packaging. Accordingly, the packaging would contain a complete cycle of pills, for example, 28 days or 30 or 31 days, wherein the cycle could contain vanoxerine in differing concentrations, as well as placebo pills on some days to provide for effective treatment of the cardiac arrhythmia.

In other embodiments and methods of administration, an initial dose, a loading phase, and a maintenance phase may all be administered via different mechanisms. For example, a patient may be administered an initial dose in IV or as a parenteral bolus injection. The loading phase may be via an infusion device, either implanted or carried with the patient, and the maintenance phase may be with an oral formulation. The particular mode of administration, accordingly, may be altered in one or more of the phases as is appropriate for the particular patient and treatment scenario.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

28 patients participated in a study of vanoxerine. 25 patients took a 300 mg dose of vanoxerine and 3 patients took a placebo. Each patient gave blood samples before administration of their dose, and then again at nine further time points, 30 minutes after administration, 1, 2, 3, 4, 6, 8, 12, and 24 hours post administration.

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1) represents an amount that was below the lower limit of quantitation, which is <1.139 ng/ml vanoxerine, and <1.1141 ng/ml 17-hydroxyl vanoxerine.

Table 2 shows the standard deviations from the above 25 patients receiving vanoxerine. The three patients receiving a placebo are not included in the data and all data points indicated levels of vanoxerine below the lower limit of quantitation.

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider. The standard deviations in Table 4 are lower than those in Table 6, (no T-test or 95% confidence was run), demonstrating that the variability was greater in the high group than the low group.

### Example 2

12 subjects received daily doses of vanoxerine for 11 consecutive days, at doses of 25, 50, 75, and 100 mg, with a 14 day washout period between dose levels.

At 25 mg, plasma levels were not detectable after 8 hours. At 50, 75, and 100 mg doses, plasma levels were detectable at 24 hours and steady state was reached by day 8. PK was linear and the dose proportional across 50, 75 and 100 mg doses. The 100 mg QD Cₘₐₓₛₛ and AUC₀₋₂₄ₛₛ suggests a trend toward non-linear PK that may become apparent at doses >100 mg QD. PK was highly variable at steady state; Cₘₐₓ, ss, and AUC₀₋₂₄ₛₛ inter-subject variability ranged from 55-85%. The results are listed below in Table 7.

TABLE 7

| | PK Data | |
|---|---|---|
| | (Mean +/− SD) | PK Data (Mean +/− SD) |
| Dose | C_{Max} | T_{1/2} |
| 50 mg | 27.5 +/21.3 ng/ml | 49.39 +/− 26.18 hr (4.71-110.57) |
| T_{Max} 1.27 +− | | |
| 0.5 hr (0.5-2.0) | | |
| 75 mg | 27.4 +/− 15.5 ng/ml | 52.53 +/− 37.46 (10.26-116.67) |
| 100 mg | 40.2 +/− 26.6 ng/ml | 15.38 +/− 43.55 (5.56-125.00) |

Data from these studies demonstrates an increased half-life of the drug when daily doses are given. Furthermore, it was noted that heart rate and systolic blood pressure increased slightly in most subjects at 75 and 100 mg doses and did not completely return to baseline during washout between dose levels.

### Example 3

Fourteen healthy patients were given vanoxerine at 25, 75, and 125 mg, daily, for 14 days with a washout of 14 days between dose levels. A standardized meal was served 15 minutes prior to each dosing.

No significant adverse events were seen in any of the studies. Steady state serum levels were reported within 9-11 days with disproportionately and statistically greater levels at higher doses as compared with the lower doses. The non-linear kinetics may be due to increasing bioavailability at higher doses based on a saturation of first pass metabolism.

### Example 4

Four patients were given 50, 100, and 150 mg vanoxerine, daily, for 7 days.

Upon administration of 100 mg for 7 days, increases in systolic blood pressure and heart rate were seen. Similarly, during the 150 mg test, the patients also saw increases in systolic blood pressure and in heart rate. Steady-state levels were achieved within one week for all patients.

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of the study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

As described herein, a loading phase includes administration of vanoxerine of about 25 to about 300 mg a day taken daily for about 3 to about 14 days. In some embodiments, the loading phase is met upon reaching steady state, which can be identified through blood samples from a patient. The steady state levels are a mean plasma concentration of about 1 to about 200 ng/ml, about 5 to about 200 ng/ml, about 10 to about 200 ng/ml, and preferably about 20 to about 150 ng/ml, or about 20 to about 125 ng/ml. Furthermore, it may be most effective to provide for the minimum dose of vanoxerine over a given time period for achieving steady state, followed by subsequent administration of vanoxerine to maintain such steady state. Accordingly, because of the long half-life, a method of administration of dosing vanoxerine comprises a loading dose of the drug until steady state is met followed by subsequent vanoxerine administration about every 24, 48, or 72 hours to maintain therapeutic blood levels without the adverse effects of increased systolic blood pressure or heart rate. Furthermore, and anti-anginal compound can be administered in the maintenance phase to aid in preventing re-occurrence of arrhythmia.

Furthermore, in other embodiments, it may be advantageous to first begin a loading phase of vanoxerine to reach steady state followed by a maintenance phase to maintain the therapeutic levels of vanoxerine by administration of daily ranolazine or other anti-anginal compound and vanoxerine taken on a reduced schedule every 48 hours, every 72 hours, every 96 hours, or more, wherein the therapeutic levels of vanoxerine are maintained.

In particular, it may be advantageous to determine the profile of the patient because of the known variability with vanoxerine such that the schedule for subsequent administration of vanoxerine post the loading phase is determined by the pharmacokinetic profile of the individual patient. Accordingly, the method comprises administration of vanoxerine in a patient to meet a pre-determined steady state therapeutic level; determination of the patient's half-life once steady state has been reached, and followed by a dosing regimen comprising ranolazine or another anti-anginal drug taken daily (or as prescribed) and vanoxerine taken according to the determined half-life profile of the patient. Thereby, the patient takes the minimum vanoxerine needed to maintain therapeutic levels to prevent recurrence of cardiac arrhythmia and to maintain normal sinus rhythm.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A pharmaceutical composition comprising an effective amount of vanoxerine, an antianginal compound, and a pharmaceutical carrier, which is suitable for administration to a mammal for treatment of cardiac arrhythmia.

2. The pharmaceutical composition of claim 1 wherein the antianginal compound is ranolazine.

3. The composition of claim 1 wherein the effective amount of vanoxerine is between 25 and 400 mg.

4. The composition of claim 1 wherein the effective amount of vanoxerine provides for a plasma concentration of between 20 and 200 at a time between 1-4 hours post administration.

5. A method for maintaining a plasma level concentrations in a patient being treated for cardiac arrhythmia comprising: administering an effective amount of vanoxerine to reach steady state; upon reaching steady state, administering an effective amount of vanoxerine to maintain steady state and further administering an anti-anginal compound to said patient subsequent to reaching steady state.

6. The method of claim 5 wherein said steady-state concentration is about 10-200 ng/ml as measured in the plasma of a mammal.

7. The method of claim 5 wherein said steady-state concentration is about 20-125 ng/ml as measured in the plasma of a mammal.

8. The method of claim 5 wherein the vanoxerine and anti-anginal compound are administered in alternating doses to maintain steady state.

9. A method of treating a patient suffering from cardiac arrhythmia comprising a loading phase and a maintenance phase:
a. identifying a patient experiencing an episode of cardiac arrhythmia;
b. during the loading phase, administering vanoxerine to said patient for induction of steady state pharmacological concentration; and
c. during the maintenance phase, administering an antianginal compound subsequent to induction of steady state pharmacological concentration, and administering a further dose of vanoxerine to said patient to maintain steady state pharmacological concentration.

10. The method of claim 9 wherein said loading phase is about 3 to about 10 days.

11. The method of claim 9 wherein said loading phase is about 7 days.

12. The method of claim 9 wherein said loading phase is about 14 days.

13. The method of claim 9 wherein said steady state concentration is about 10-200 ng/ml as measured in the plasma of a mammal.

14. The method of claim 9 wherein said steady state concentration is about 20-125 ng/ml as measured in the plasma of a mammal.

15. A method of chronic administration of vanoxerine comprising a loading phase and a maintenance phase, wherein said loading phase comprising administration of about 25 to 200 mg of vanoxerine daily until steady state concentrations are met; and wherein said maintenance phase comprises administration of vanoxerine to maintain said steady state concentration, and wherein said maintenance phase further comprises at least one additional anti-arrhythmic drug administered to said same patient.

16. The method of claim 15 wherein said loading phase is about 3 to about 10 days.

17. The method of claim 15 wherein said loading phase is about 7 days.

18. The method of claim 15 wherein said loading phase is about 14 days.

19. The method of claim 15 wherein said steady state concentration is about 10-200 ng/ml as measured as mean AUC in the plasma of a mammal.

20. The method of claim 15 wherein said steady state concentration is about 20-125 ng/ml as measured as mean AUC in the plasma of a mammal.
